(19) United States
(12) Patent Application Publication
(10) Pub. No.: US 2010/0104619 A1
(43) Pub. Date: Apr. 29, 2010

(54) DELIVERY SYSTEM FOR A NON-STEROIDAL NON-IONIZED HYDROPHILIC DRUG

(75) Inventors: Wouter De Graaff, Oss (NL); Armin Szegedi, New York, NY (US)

Correspondence Address: ORGANON USA, INC.;c/o MERCK, 2000 Galloping Hill Road, Mail Stop: K-6-1, 1990, Kenilworth, NJ, 07033, US

(73) Assignee: N.V. ORGANON, Oss (NL)

(21) Appl. No.: 12/515,897

(22) PCT Filed: Nov. 21, 2007
(86) PCT No.: PCT/EP2007/062626

§ 371 Date: Dec. 18, 2009

(30) Foreign Application Priority Data
Nov. 22, 2006 (EP) 06124598.1

(51) Int. Cl. A61K 9/00; A61K 47/32; A61P 43/00

(52) U.S. Cl. 424/430; 514/772.4

(57) ABSTRACT

This invention relates to an extended release formulation comprising a solid non-steroidal non-ionized hydrophilic drug, having a molecular weight below 500 Dalton and having a solubility of at least 0.1 wt % in ethylene vinyl acetate copolymer having a vinyl acetate content of 28%, which formulation is a vaginal device having a skin and which device comprises an inner compartment made of a thermoplastic polymer, which polymer is containing the drug. The polymer is preferably made of ethylene-vinyl acetate copolymer.

The present invention relates to an extended release formulation comprising a solid non-steroidal non-ionized hydrophilic drug, having a molecular weight below 500 Dalton.

Solid non-steroidal non-ionized hydrophilic drugs, having a molecular weight below 500 Dalton are widely used in many therapeutic areas with dosage regimes for daily intake of tablets. With such dosage regimes based on prescription of tablets which have to be taken daily, it is very common that tablets are forgotten and that compliance of the patient with the treatment is less than desired. There is therefore a strong need for a patient friendly extended release formulation of drugs. In general, there are many extended release formulations available and most of them are based on implantation or injection of the formulation. Alternatives are patches for transdermal delivery. As an example of an injection formulation is long-acting risperidone (Risperdal Consta®). Risperdal Consta® is an aqueous suspension of microspheres comprising risperidone and a biodegradable copolymer for intramuscular administration with incidence of injection site pain. The method for preparing the microspheres having a substantial sigmoidal release profile with an initial lag phase is described in U.S. Pat. No. 6,596,316. The typical starting dose of Risperdal Consta® is 25 mg every 2 weeks. Depending on an individual's response, the dose can be increased to a maximum of 50 mg every 2 weeks. Currently, Risperdal Consta® is available in strengths of 12.5 mg, 25 mg, 37.5 mg and 50 mg injections. The product causes less plasma drug fluctuation than the oral formulation. Significant release of risperidone from the microspheres begins 3 weeks after the first injection, thus administration of an oral antipsychotic is necessary during this period. Steady-state plasma concentrations are reached after the fourth injection. Effects of long-acting risperidone persist for at least two weeks after injection, the time period needed for degradation of the microspheres comprising the risperidone. Obviously, medication of the patients cannot be interrupted in this period of two weeks in case of side-effects. Elimination is complete 7-8 weeks after the last injection (Harrison, T. S., and Goa, K. L. Long-acting risperidone: review of its use in schizophrenia. CNS Drugs (2004), 18:113-132).

US 2003/0153983 describes implantable medical devices that provide resistance to microbial growth on and in the environment of the device and resistance to microbial adhesion on the device. In U.S. Pat. No. 4,469,671 a contraceptive device for intravaginal use is described comprising a bioinsoluble, biocompatible polyurethane and an acrosin inhibitor such as salts of alkyl or alkenyl sulfate. An elastomeric vaginal ring comprising a pharmacologically active compound or pharmaceutically acceptable addition salts for the treatment of cancer is described in U.S. Pat. No. 5,558,877. An elastomeric matrix type of system for vaginal delivery of antimicrobial agents is described in WO 02/076426. U.S. Pat. No. 4,016,251 discloses a drug-delivery device comprising of a shaped body of ethylene-vinyl acetate containing a drug and permeable to passage of the drug by diffusion.

In some fields like psychiatric drug treatment it is highly unusual to contemplate a vaginal delivery system for extended release (WO 03/055424). Rather, this route of administration appears acceptable for hormonal contraceptive regimes or hormone replacement therapies which are exclusively aimed at treatment of the female person. In general, vaginal delivery devices are well-known in the field of gynaecology for the delivery of hydrophobic steroidal drugs for contraceptive uses, such as exemplified in U.S. Pat. No. 4,292,965, WO97/02015, WO2004/103336 and EP 0 876 815. A contraceptive vaginal ring is marketed under the trademark Nuvaring® by Organon, the Netherlands. Such rings are designed for the purpose of administering high potency steroids, for which drug delivery rates in the order of 0.01 to 0.5 mg/day are usually sufficient to obtain beneficial therapeutic effects. However, for solid non-steroidal non-ionized hydrophilic drugs, having a molecular weight below 500 Dalton, therapeutically effective amounts to be delivered locally is much higher and usually ranges in the order of 0.1-60 milligrams a day. Although it is uncommon to manufacture drug formulations for female use only of drugs which are not uniquely meant for female users, it nevertheless is of great advantage to provide an extended release formulation for these hydrophilic drugs in the form of a vaginal delivery device. As an example, mirtazapine is used for therapeutic indications which occur more frequently in women, so that an extended release formulation which can only be used for women is still an important contribution to the art. For example, another drug for the treatment of depression, fluoxetineHCl, has been proposed for use in an extended release formulation in the form of a vaginal delivery system (WO 03/055424). The described device includes one or more channels in the surface for receiving a drug formulation, a pocket molded in the ring to receive a drug formulation or comprises a hollow toroid polydimethylsiloxane tubing for use of higher dose delivery. WO 2005/004837 describes a device with a reservoir containing dispersed active agent and a sheath discontinuously surrounding the reservoir. WO0170154 discloses a siloxane elastomer vaginal ring device with a bore located in the ring comprising an oxybutynin composition, wherein the bore runs from the surface of the ring into the ring. For non-steroidal drugs the choice for polysiloxane polymers relates to their high drug solubility and the well known high permeability of polysiloxane polymers (A. D. Woolfson, R. K. Malcolm, R. J. Gallagher, Journal of Controlled Release 91 (2003) 465-476). In addition, the diffusion coefficient for the same type of molecules in polysiloxanes is typically 100 to 200 times higher than the diffusion coefficient found in polyvinyl acetate copolymers (poly-EVA) (Treatise on controlled drug delivery; fundamentals, optimization, applications, edited by A. Kydonieus, Marcel Dekker Inc. New York, 1992. Typical diffusion coefficient for steroids, pp. 66-67).

Unexpectedly, it has now been found that an extended release formulation in the form of a vaginal delivery system can be prepared for non-steroidal non-ionized hydrophilic drugs, having a molecular weight below 500 Dalton, with superior drug delivery characteristics in terms of high-release rate of drug, almost lacking initial burst release, substantially constant release rate, in combination with a high drug substance efficiency and a duration of use of from one week up to 1 month, and which has optimal mechanical properties, in particular flexibility in the delivery system according to the invention by avoiding the use of polysiloxane as taught in the prior art.

The present invention provides for a vaginal device comprising a solid non-steroidal non-ionized hydrophilic drug, having a molecular weight below 500 Dalton and having a solubility of at least 0.1 wt % in ethylene vinyl acetate copolymer having a vinyl acetate content of 28%, a skin and an inner compartment, which inner compartment is made of a thermoplastic polymer, which polymer is containing the drug. The inner compartment does not contain a hollow structure like tubings. Solubility is measured as described in Laarhoven, J. A. H van, et al. (2002), International Journal of Pharmaceutics 232, page 165. Preferably, the skin is substantially a continuous cover over the inner compartment. Good results can be obtained when the inner compartment contains 5-80 wt % of the hydrophilic drug. Preferably, the inner compartment comprises a core, which does not contain solid hydrophilic drug. Preferably, the inner compartment, and/or the skin, and/or the core or all three of these is or are made of ethylene-vinyl acetate copolymer. In a more specific embodiment an ethylene-vinyl acetate copolymer having a vinyl acetate content in the range of 6 to 40% is used.

Advantageous characteristics of the invention are that the device can easily be manufactured using extrusion techniques and is flexible in view of the small cross-sectional diameter if manufactured in the form of a ring. In addition to that, the extended release formulation according to the invention has an intrinsically safe design against dumping of the high dose. By application of a core in the inner compartment, the system allows for an improved release efficiency. Application of a core also allows to tune mechanical properties of the system which are relevant in relation to comfort (foreign body feeling) and retention without affecting release kinetics significantly.

An extended release formulation according to the invention compared with administration by injection has the advantage of non-invasive administration, of providing drug release immediately upon exposure of the formulation to aqueous media, of immediate interruption of drug delivery after removal of the system from the vagina, which is particularly advantageous in case medical practitioners have the incentive to interrupt or change the treatment for reasons related to insufficient therapeutic effect or to serious adverse effects during treatment.

The presence of the hydrophilic drug in solid form provides for a sufficient and continuous supply of the drug during release and the solid form prevents crystallisation of the drug on the outside of the device during manufacturing.

### Clarification of Terminology.

For clarity and/or to define more specific embodiments of the invention the term ‘hydrophilic’ drug may be further specified as a drug having a contact angle of less than 90, or less than 60, or less than 50, or less than 40, or less than 30, or less than 20 degrees, determined as described in C. F. Lerk, et. al, J. Pharm. Sci. (1977), 66:1480. With non-ionized drug is meant drug in the free-base form. Salts of drugs are not suitable for delivery by the extended release formulation according to the invention.

With a vaginal device a drug delivery system for insertion into the vagina of a woman is meant. The system has preferably the form of a ring, such that the delivery system has an elongated shape of which the two ends are joined together. The ring may comprise one or more loops and those loops may have various shapes, such as oval, ellipsoidal, toroidal, triangular, square, hexagonal, octagonal, etc. Alternatively, the system according to the invention is helically-shaped, which means the shape of a fibre helix with more than one loop and two ends which are not joined together.

With continuous skin is meant that the skin is continuously surrounding the drug containing compartment and is devoid of expressly provided parts in the skin for release of the drug. Thus, direct contact between vaginal tissue and drug compartment is minimised in order to avoid local irritation. The skin in substantially continuous in the sense that only incidental apertures may be present for example, the ends of a helically shaped system or apertures due to shear during manufacturing or due to incomplete closure of ring ends, but such openings are not purposefully introduced into the skin in order to facilitate the passage of drug through the skin. It is not excluded that the skin material may comprise some dissolved drug.

An inner compartment of the device is the compartment which contains the drug to be delivered to the patient and is covered by the skin. Therefore, there is no direct contact between the vaginal tissue and the inner compartment. The skin is the barrier protecting the vaginal tissue from undesirable local effects from the concentrated drug in the inner compartment. The inner compartment is formed by a thermoplastic polymer.

A core is an inner structure within the inner compartment and serves to reduce the drug containing space in the inner compartment. The core does not contain solid drug. It is not excluded, though, that the core material may comprise some dissolved drug. When drug is loaded into the inner compartment during the production process some drug may enter into the core. The core can be made of any suitable material such as a metal, a polymer or the same material as the polymer used for the inner compartment. The core can also contribute to the strength or flexibility of the device and to increased release efficiency. In another context the inner compartment is also referred to as an intermediate layer when a core is present in the device.

The present invention provides for delivery rates of drug in the range of 0.1 to 60 mg/day for a period of use of from one week up to 1 or 2 months.

Suitable drugs for extended release in the system according to this invention are those selected from the group consisting of vitamin K antagonists, such as fenprocoumon; drugs for coronary therapy such as nicorandil; nitrites and nitrates such as isosorbide-5-mononitraat and nitroglycerin; anticoagulants such as acenocoumarol and dipyramidol; anti-arrhythmics such as flecamide; antihypertensives such as moxonidine and minoxidil; diuretics such as triamterene and hydrochlorthiazide; betablocking agents such as atenolol; calcium antagonists such as isradipine; ACE-blocking agents such as trandolapril; trichomonacides such as tinidazol; antimycotics such as clotrimazol, miconazol, solifenacine and dinoproston; anti-inflammatory agents such as tenoxicam; anti-bacterial agents such as metronidazole and nitrofurantoin; drugs in incontinence such as and oxybutynin; local anesthetics such as levobupivacaine, lidocaine and bupivacaine; narcotic analgetics such as piritramide, fentanyl, dextromoramidum, buprenorfine and tramadol; non-narcotic/anti-pyretic drugs such as naproxen, ibuprofen and metamizol; migraine drugs such as flunarizine; anti-parkinson drugs such as biperideen, and trihexylenidyl; antipsychotics such as aripiprazole, risperidone, sertindole, olanzapine, quetiapine, benperidol, haloperidol, fluspirilene, bromperidol, tiotixene, periciazine, pimozide, pipotiazine and penfluridol; antidepressants such as paroxetine, doxepine and mirtazapine; systemic antihistaminics such as loratidine, desloratidine, stemizol, xatomide and terfenadine; anti-asthmatica such as salbutamol; anti-viral drugs_such as UC781 with chemical name N-[4-chloro-3-(3 methyl-2-butenyloxy)phenyl]-2-methyl-3-furano-carbothiamide and TMC120; oncology drugs such as cis-platin.

It is a specific embodiment of the invention to provide for a delivery system as described above comprising a vitamin K antagonist, such as fenprocoumon. It is a specific embodiment of the invention to provide for a delivery system as described above comprising a drug for coronary therapy such as nicorandil. It is a specific embodiment of the invention to provide for a delivery system as described above comprising a nitrite or nitrate such as isosorbide-5-mononitraat and nitroglycerin. It is a specific embodiment of the invention to provide for a delivery system as described above comprising an anticoagulant such as acenocoumarol and dipyramidol. It is a specific embodiment of the invention to provide for a delivery system as described above comprising an anti-arhitmic such as flecamide.

It is a specific embodiment of the invention to provide for a delivery system as described above comprising an antihypertensive such as moxonidine and minoxidil. It is a specific embodiment of the invention to provide for a delivery system as described above comprising a diuretic such as triamterene and hydrochlorthiazide. It is a specific embodiment of the invention to provide for a delivery system as described above comprising a beta-blocking agent such as atenolol. It is a specific embodiment of the invention to provide for a delivery system as described above comprising a calcium antagonist such as isradipine. It is a specific embodiment of the invention to provide for a delivery system as described above comprising an ACE-blocking agents such as trandolapril. It is a specific embodiment of the invention to provide for a delivery system as described above comprising a trichomonacide such as tinidazol.

Thus, it is a specific embodiment of the invention to provide for a delivery system as described above comprising an antimycotic such as clotrimazol, miconazol, solifenacine and dinoproston. It is a specific embodiment of the invention to provide for a delivery system as described above comprising an anti-inflammatory agent such as tenoxicam. It is a specific embodiment of the invention to provide for a delivery system as described above comprising anti-bacterial agent such as metronidazole and nitrofurantoin. It is a specific embodiment of the invention to provide for a delivery system as described above comprising a drug against incontinence such as tolterodine and, in particular oxybutynin. It is a specific embodiment of the invention to provide for a delivery system as described above comprising a local anesthetic such as levobupivacaine, lidocaine and bupivacaine. It is a specific embodiment of the invention to provide for a delivery system as described above comprising a narcotic analgetic such as piritramide, fentanyl, dextromoramidum, buprenorfine and tramadol. It is a specific embodiment of the invention to provide for a delivery system as described above comprising a non-narcotic/anti-pyretic drug such as naproxen, ibuprofen and metamizol. It is a specific embodiment of the invention to provide for a delivery system as described above comprising an antimigraine drug such as flunarizine. It is a specific embodiment of the invention to provide for a delivery system as described above comprising an anti-parkinson drug such as biperideen, and trihexylenidyl. It is a specific embodiment of the invention to provide for a delivery system as described above comprising an antipsychotic such as aripiprazole, risperidone, sertindole, olanzapine, quetiapine, benperidol, haloperidol, fluspirilene, bromperidol, tiotixene, periciazine, pimozide, pipotiazine and penfluridol. It is a specific embodiment of the invention to provide for a delivery system as described above comprising an antidepressant such as doxepine, mirtazapine and in particular paroxetine. It is a specific embodiment of the invention to provide for a delivery system as described above comprising a systemic antihistaminic such as loratidine, desloratidine, stemizol, xatomide and terfenadine. It is a specific embodiment of the invention to provide for a delivery system as described above comprising an anti-asthmatic such as salbutamol.

It is a specific embodiment of the invention to provide for a delivery system as described above comprising an anti-viral drug such as UC781 with chemical name N-[4-chloro-3-(3-methyl-2-butenyloxy)phenyl]-2-methyl-3-furano-carbothiamide, TMC120 (dapivirine) and tenovir. It is a specific embodiment of the invention to provide for a delivery system as described above comprising an oncology drug such as cis-platin.

The characteristic of the invention may be understood and influenced by the following explanation and use thereof: Fick's law of diffusion governs the release of compounds. Vaginal rings are cylindrical reservoir/membrane designs of which the release rate can be described by the equation below. Suitable rings can therefore be made by an appropriate choice of the parameters that affect the release rate.

The release rate of a cylindrical reservoir/membrane design is:

$$\frac{d M}{d t} = \frac{2 π L {D}_{p} {K}_{p / s} Δ C}{Ln ( {r}_{0} / {r}_{i} )}$$

L=the length of the cylinder
Dₚ=the diffusion co-efficient of the compound in a skin polymer
K_{p/s}=partition coefficient of the compound between the skin and inner compartment
ΔC=the difference in concentration of dissolved drug between the inner compartment near the skin and the sink
r₀=is the overall radius, i.e. the cross-sectional diameter including the skin
rᵢ=is the radius of the inner compartment (i.e. r₂/r₁=1) or of the core plus inner compartment (i.e. r₁, core comprising ring)

The equation shows that zero order release is obtained when the term on the right-hand side of the equation is constant, i.e. not a function of time.

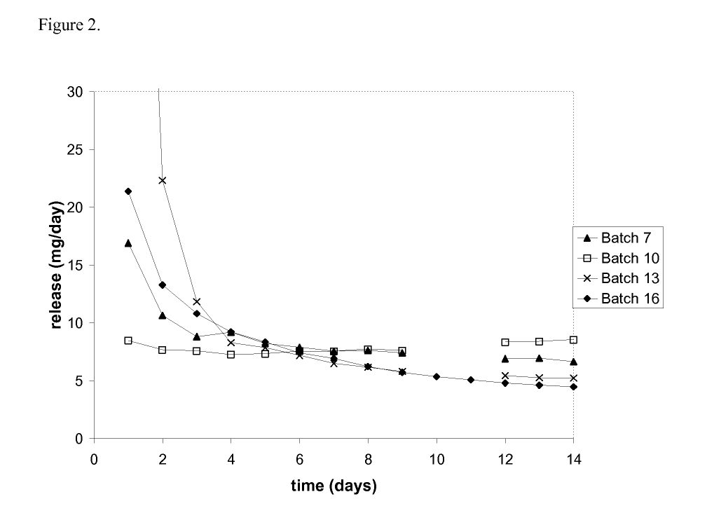

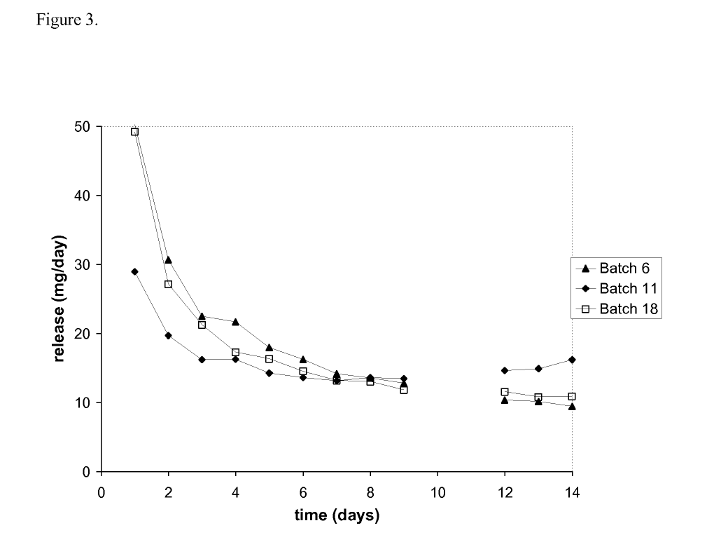

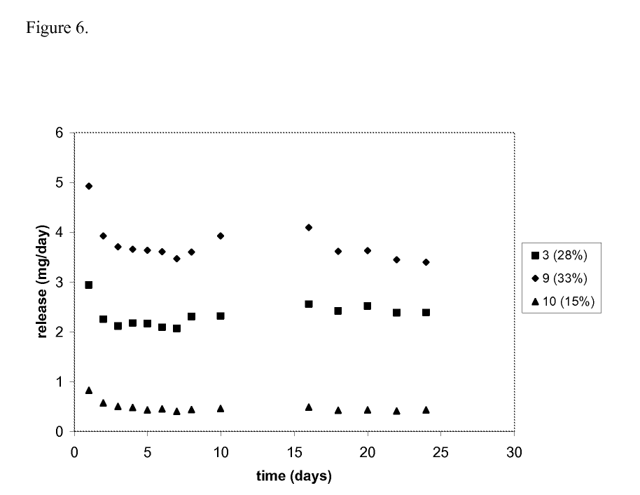

It is shown in FIGS. 2 and 3 that release rates of mirtazapine of 7.5 to 25 mg/day and in FIGS. 6 to 8 that release rates of risperidone of 0.4 to approximately 4-5 mg/day can be achieved with the devices according to the invention having a skin substantially continuously covering the inner compartment.

Apparently, as an example drugs and specific embodiments the solubility of mirtazapine and risperidone in ethylene-vinyl acetate (EVA) of the inner compartment is such that the ΔC for the drugs is high enough to provide for fast release kinetics. The limiting factor in maintaining a substantially constant ΔC in a quasi steady state with a high release rate of the drugs, i.e. maintaining a substantially constant drug delivery from the device in the presence of a relatively thin skin with low barrier properties, is the supply of dissolved drug to the interface between the inner compartment and the skin. The supply (or referred to as release rate) is the result of a complex mass transport process determined by factors including the dissolution rate of the drug into the polymer, which in turn is determined by the solubility of the drug in the polymer and the surface area of the drug exposed to the polymer. The latter is determined by particle size, shape and drug content. Also the diffusion rate of the drug through the polymer is an important factor for the dissolution and release rate. It has been found that devices having about 40 to 80 wt % of mirtazapine or risperidone in the inner compartment not only provide for fast release rates but, when compared with devices comprising 5 to about 40%, in addition to that, provide for significantly more linear or substantially constant release kinetics.

It is believed that with drug contents in the polymer above 40 wt. % drug particles can be close to each other within the polymer of the inner compartment. The structure formed by the dispersed solid particles in the polymer depends on drug content and additionally on particle size and shape. During drug release, the properties of the inner compartment itself change in time by the slow dissolution of the drug particles, apparently facilitating drug dissolution and transport rate resulting substantially constant high release rates. Probably the formation of improved diffusion pathways in the polymer by the progressively dissolving particles leaving voids in the polymer and the simultaneous flow of aqueous liquids through the skin into the inner compartment filling the voids with water are important factors in achieving substantially constant release at high levels of drug content.

In the delivery devices of the invention drug is present in all polymer layers. When a drug in the manufacturing process of the system is loaded into the inner compartment, the drug diffuses during the production process and/or during storage of the system to the other polymer layer(s) up to equilibrium concentration.

In line with the concept of the core comprising ring, for a ring without core the lengthening of the diffusion distance should also be kept as small as possible and the active compound should also be present in the solid form in order to obtain essentially zero-order release kinetics. Lengthening of the diffusion distance in case of the ring without core can be kept relatively small by keeping the cross-sectional diameter of the inner compartment relatively small. Such a small diameter also results in a relatively small volume of the inner compartment and hence, the amount of active compound, which is required to sustain the release for the intended period of use, is loaded in high concentration in the inner compartment.

A high concentration of active compound in the inner compartment of a ring without core also could be achieved in a large diameter ring, but this would require the use of a large excess of active compound, i.e. much more than required to sustain the release over the intended period of use and hence, this results in an economically and environmentally less attractive dose form with a low release efficiency.

In analogy with a small inner compartment volume of the ring without core, a small inner compartment volume of the core comprising ring serves the purpose of concentrating the active compound in a relatively small polymer volume during processing.

The vaginal delivery system according to the present invention can provide a release rate of drug in the range of 0.1 to 60 mg/day for a period of use of from one week up to 1 month. Preferably the rate is in the range of 0.5 to 20 mg/day, most preferably in the range of 2 to 20 mg/day.

The thermoplastic polymer that can be used in making the drug delivery system according to the present invention may in principle be any extrudable thermoplastic polymer material suitable for pharmaceutical use, such as ethylene-vinyl acetate (EVA) copolymers, low density polyethylene, polyurethanes, and styrene-butadiene copolymers. In a preferred embodiment, ethylene-vinyl acetate copolymer is used due to its excellent mechanical and physical properties. The EVA copolymer may be used for the core, the intermediate compartment (inner compartment) as well as the skin and can be any commercially available ethylene-vinyl acetate copolymer, such as the products available under the trade names: Elvax, Evatane, Lupolen, Movriton, Ultrathene, Ateva, and Vestypar. These ethylene-vinyl acetate copolymers are available in different grades with respect to the amount of vinyl acetate present in the copolymer, for example, EVA 28 is a copolymer having a vinyl acetate content of 28%.

In one embodiment, at least the skin is made of ethylene-vinyl acetate copolymer. In a further embodiment, the core, the inner compartment, and the skin or the inner compartment and the skin (in a ring without core) are made of ethylene-vinyl acetate copolymers, which copolymers can each be of the same or different grades.

In another embodiment, the inner compartments are made of the same grade of ethylene-vinyl acetate copolymer. However, by electing different polymer grades for the inner compartment, fine-tuning of the flexibility of the ring is possible. The thickness of the skin and the vinyl acetate content of the skin influence the release rate of the active ingredient. The thinner the skin and the higher the vinyl acetate content of the skin, the higher the release rate of the active ingredient.

In one embodiment, EVA copolymers having a vinyl acetate content of from 6% to 40% are used. In another embodiment, EVA copolymers having a vinyl acetate content of from 6% to 33% are used. In a further embodiment, EVA copolymers having a vinyl acetate content of from 6% to 28% are used. In yet another embodiment, EVA copolymers having a vinyl acetate content of from 9% to 28% are used. In a further embodiment, the core is made of EVA 28 or 33. In another embodiment, the skin is made of EVA copolymers having a vinyl acetate content of from 6% to 28%. In yet another embodiment, the skin is made of EVA copolymers having a vinyl acetate content of from 9% to 28%, for example, EVA 9, EVA 15, EVA 18, EVA 28 or EVA 33. It is known in the art that the lower the vinyl acetate content of the EVA copolymers used, the higher the stiffness of the vaginal ring made thereof. Moreover, a larger cross-sectional diameter will also result in a higher stiffness, i.e. less flexibility.

A vaginal ring of the present invention can be manufactured by the known process of extrusion, such as co-extrusion and blend extrusion. To obtain the material for the inner compartment comprising the drug, the drug is mixed with an EVA copolymer. The major step in the mixing process is blend extrusion. Subsequently, the drug/EVA copolymer mixture is co-extruded with the core and skin materials into a three-layered (core comprising) fibre. Alternatively, the drug/EVA copolymer mixture is co-extruded with the skin material into a two-layered fibre (ring without core). After this step, the drug will partly be dissolved in the EVA copolymer. The solubility of the drug in the copolymer is determined by the vinyl acetate content of the EVA copolymer used. Any drug material that is not dissolved will be present as a solid phase in the .inner compartment The solid phase will be in equilibrium with the dissolved phase of the drug, such providing a constant concentration of dissolved active substance close to the rate controlling skin layer. The three-layered or two-layered fibre thus-obtained is cut into pieces of a desired length and each piece is assembled to a ring-shaped device in any suitable manner known to the person skilled in this art. The rings are then packed, for example in a suitable sachet, optionally after being sterilized or disinfected.

A person skilled in the art of extrusion will have no difficulty in finding the optimal processing conditions, such as determining the extrusion temperature, extrusion speed, and air gap, for making a three-layered or two-layered fibre containing drug on the basis of methods and procedures known in the art and the description and examples given in this application. A suitable temperature for blend extrusion of the mirtazapine/EVA copolymer mixture lies in the range of from 80° C. to 110° C., e.g. approx. 100° C. Suitable temperatures for co-extrusion of the three-layered or two-layered fibre lie in the range of from 80° C. to 110° C., e.g. from 90° C. to 110° C. A suitable temperature for blend extrusion of risperidone/EVA copolymer mixture lies in the range of from 80° C. to 140° C., e.g. approx. 90° C. Suitable temperatures for co-extrusion of the three-layered or two-layered fibre lie in the range of from 80° C. to 140° C.

A preferred temperature for extrusion of drug/EVA copolymer mixtures is below the melting point of the drug. i.e. below 120° C. for mirtazapine and below 170° C. for risperidone. Melting the drug during extrusion may lead to phenomena like delayed crystallization of the drug. In the manufacture of the extended release formulation according to the invention the crystalline form of the solid non-steroidal non-ionized hydrophilic drug is preferred.

In this way, vaginal rings with constant release rates of drug, for example releasing in the range of 0.1 to 60 mg/day of drug, can be manufactured.

The vaginal ring according to the present invention can be manufactured in any practical size. In one embodiment, the ring has an outer diameter of between about 50 and 60 mm and in another embodiment between about 52 and 56 mm. In a further embodiment, the cross-sectional diameter is between about 2.0 and 6.0 mm, in a still further embodiment between about 2.5 and 5.0 mm, in another embodiment between about 3.0 and 4.5 mm, and in yet another embodiment it is about 4.0 mm.

In one embodiment, the amount of drug contained in the inner compartment is from 5 to 80 wt %, in another embodiment from 10 to 70 wt %, in still another embodiment from 30 to 70 wt %, and in a further embodiment from 40-65 wt %, and in yet another embodiment from 55-65 wt %.

In another embodiment, the skin is made of EVA copolymers having a vinyl acetate content of from 9% to 28% and the amount of drug contained in the medicated inner compartment is 40-65 wt %. In yet another embodiment, the skin is made of EVA copolymers having a vinyl acetate content of from 15% to 33%, a thickness in the range of 30 to 200 μm, the copolymer of the inner compartment contains 28 to 33 wt % of vinylacetate and the amount of drug contained in the medicated inner compartment is 30-65 wt %.

In one embodiment the drug delivery system according to the invention is a cylindrical fibre, consisting of a cylindrical inner compartment and a skin covering this compartment. In a particular embodiment the cross sectional diameter of such a cylindrical fibre is between about 2.5 and 6 mm, in a specific embodiment between about 3.0 and 5.5 mm, and in another embodiment between about 3.5 and 4.5 mm and in yet another embodiment is 4.0 or 5.0 mm. In one embodiment, the surface of the fibre is more than 800 mm², and in another embodiment more than 1000 mm² and in a further embodiment in the order of 1700-2200 mm². Significantly larger surfaces are possible, provided that the design (physical dimensions) of a drug delivery system intended for vaginal use prevents inconvenience for the subject.

In one embodiment said skin has a thickness in the range of 20 to 200 μm, in another 20 to 100 μm. In a still further embodiment said skin has a thickness in the range of 20 to 70 μm. In a still even further embodiment the copolymer of the inner compartment contains 18 to 33 wt % of vinyl cetate. In an even further embodiment the copolymer of the inner compartment contains 28 to 33 wt % of vinyl acetate. In an even further embodiment the copolymer of the inner compartment comprises 33 wt % of vinyl acetate.

The subject invention provides a method of manufacturing the three-layered drug delivery system of the subject invention with drug in the intermediate layer, comprising:
(i) producing a medicated homogenous polymer intermediate layer granulate;
(ii) co-extruding a polymer core granulate and the intermediate layer granulate with a polymer skin granulate to form the three-layered drug delivery system.
(iii) collecting the fibre on a reel and forming the extended release formulation according to the invention

The production of the medicated homogeneous polymer intermediate layer granulate comprises:
a. grounding the polymer;
b. dry powder mixing the grounded polymer with risperidone to be loaded in the intermediate layer;
c. blend extruding the resulting powder mixture;
d. cutting the resulting medicated polymer strands into granules, thereby obtaining an intermediate layer granulate;
e. lubricating the intermediate granulate with a lubricant.

### REFERENCES

A. Kydonieus, Marcel Dekker Inc. New York, 1992. Typical diffusion coefficient for steroids, pp. 66-67.
T. S. Harrison and K. L. Goa, Long-acting risperidone: review of its use in schizophrenia. CNS Drugs (2004), 18:113-132.
A. D. Woolfson, et al, Journal of Controlled Release (2003), 19: 465-476.
J. A. H. van Laarhoven, et al. International Journal of Pharmaceutics (2002), 232: 165.
C. F. Lerk, et. al, J. Pharm. Sci. (1977), 66:1480.

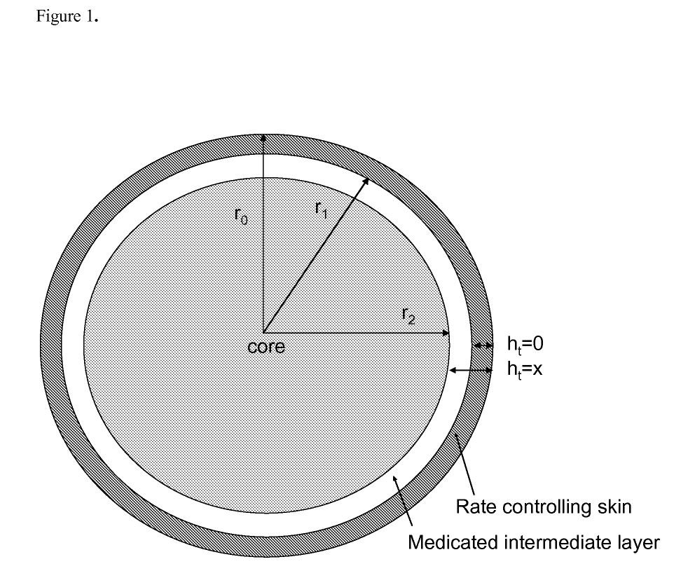

FIG. 1 shows cross-sectional presentation of a three-layered (core-comprising) vaginal delivery system according to the invention.

FIG. 2 shows the in vitro release curve of mirtazapine of three-layered rings with an average release of day 2-14 of approximately 7.5 mg/day (Batches 16, 10, 7 and 13).

FIG. 3 shows the in vitro release curve of mirtazapine of three-layered rings with an average release of day 2-14 of approximately 15 mg/day (Batches 11, 18 and 6).

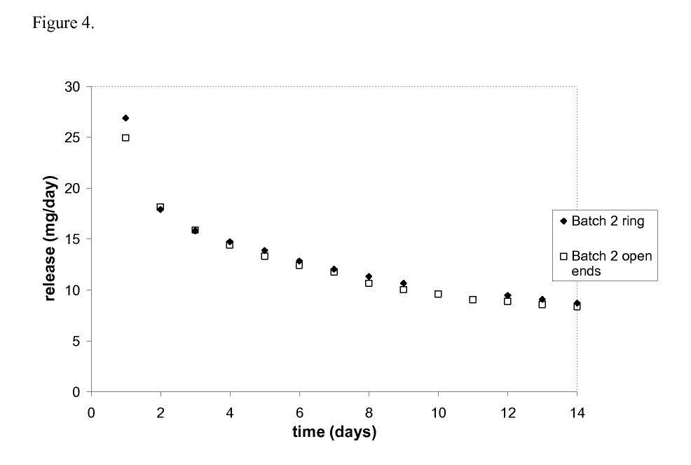

FIG. 4 shows the release rate of mirtazapine of a vaginal ring according to the invention compared with a ring, cut into a rod with two open “ring-ends” (Batch 2).

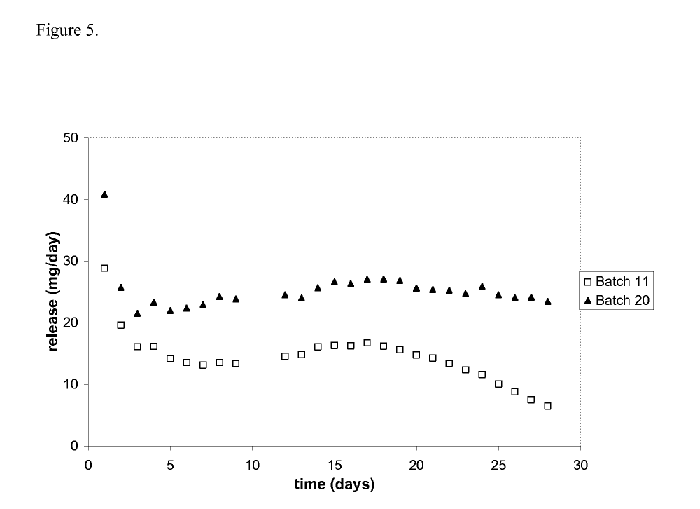

FIG. 5 shows the release rate of mirtazapine of a vaginal ring according to the invention with substantially constant release (Batches 11 and 20).

FIG. 6 shows the in vitro release rate of risperidone of vaginal rings containing with varying vinyl acetate content of the skin material (Batches 3, 9 and 10). The number in brackets refers to the wt % vinyl acetate of the copolymer.

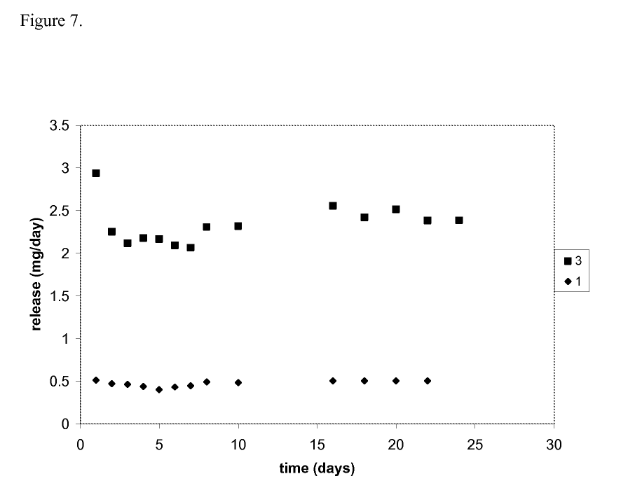

FIG. 7 shows the in vitro release rate of risperidone of vaginal rings with a skin thickness of 50 μm (Batch 3) and 200 μm (Batch 1) and EVA 28 as skin material.

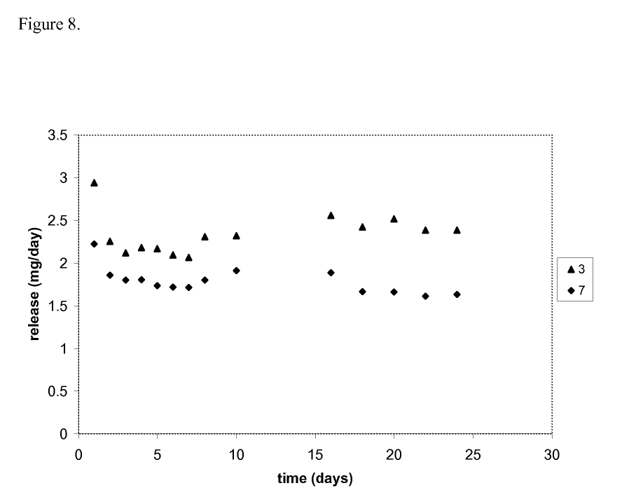

FIG. 8 shows the in vitro release of vaginal rings containing 40% (Batch 7) and 60% (Batch 3) of risperidone in the intermediate layer.

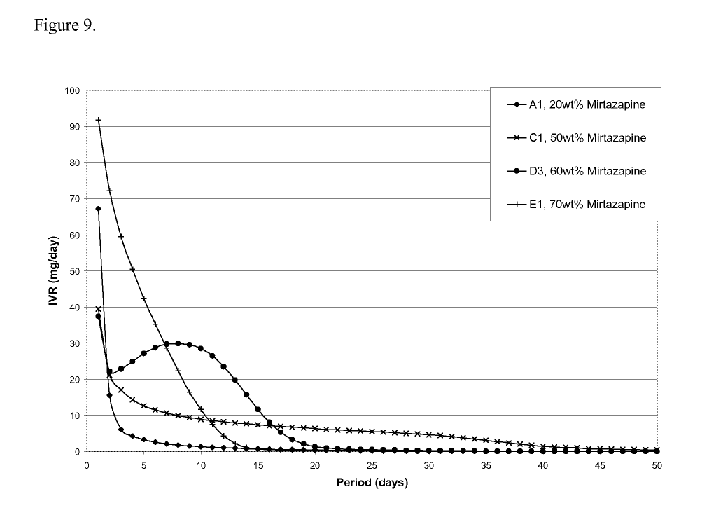

FIG. 9 shows the in vitro release rate (IVR) of mirtazapine of three-layered rings, wherein the inner compartment comprises 20 (Batch A1), 50 (Batch C1), 60 (Batch D3) and 70 wt % of drug (Batch E1) (341 μm intermediate layer thickness).

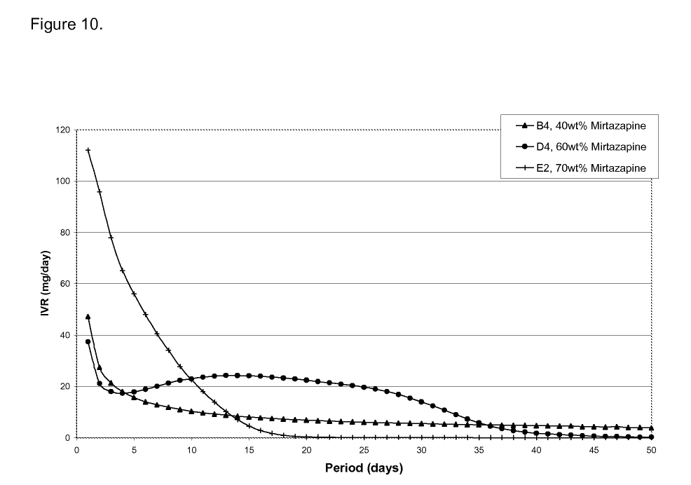

FIG. 10 shows the in vitro release rate (IVR) of mirtazapine of three-layered rings, wherein the inner compartment comprises 40 (Batch B4), 60 (Batch D4) and 70 wt % of drug (Batch E2) (682 μm intermediate layer thickness)

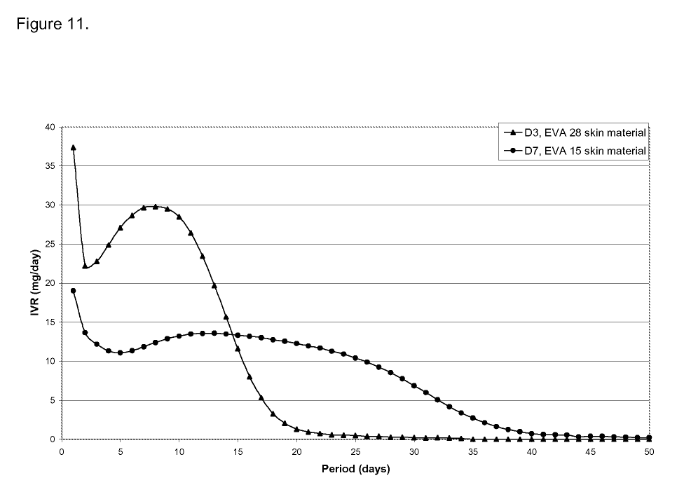

FIG. 11 shows the in vitro release rate (IVR) of mirtazapine of three-layered rings, wherein the inner compartment comprises 60 wt % of drug and the skin material is EVA 28 (Batch D3) and EVA 15 (Batch D7).

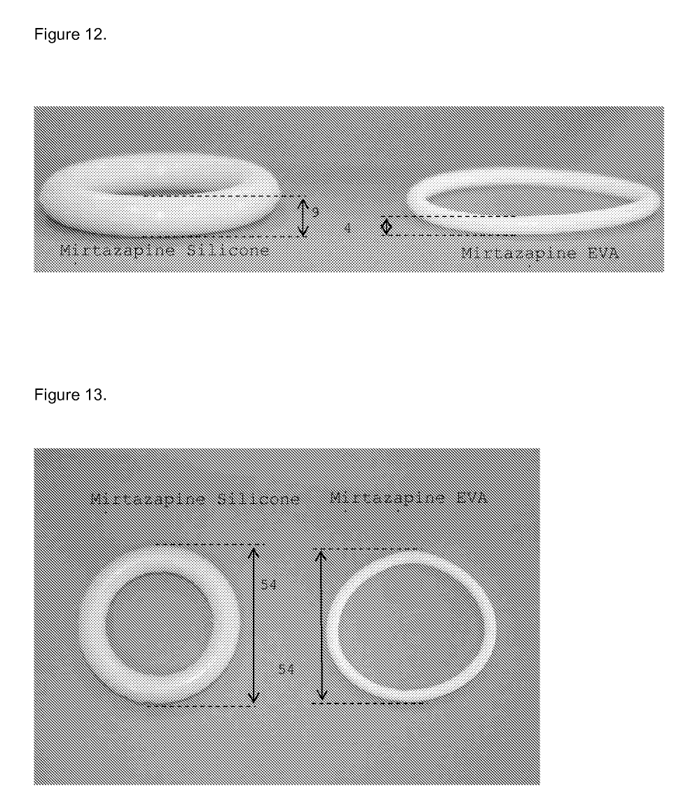

FIG. 12 shows a side-view of Silicone ring and EVA ring having a cross-sectional diameter of 9 and 4 μm respectively.

FIG. 13 shows a view from above of mirtazapine Silicone ring and mirtazapine EVA ring having an outer diameter of 54 μm.

The present invention is illustrated by the following Examples.

### EXAMPLE 1

### Preparation of Three-Layered Vaginal Rings Containing Mirtazapine

Preparation of three-layered vaginal rings consisted of several steps. First of all, an inner compartment granulate containing mirtazapine and EVA 33 copolymer was manufactured in a conventional way by pre-mixing, blend extrusion and lubrication with magnesium stearate. Secondly, a core material of EVA 28 was prepared by lubricating the as-supplied material. Subsequently, the inner compartment granulate, the core granulate and the non-medicated skin material of EVA 28 (see Table 1: A11), were co-extruded into a three-layered fibre. The fibre was cut to fibres of a specific length, as described below, after which the fibre ends were welded to a ring.

The inner compartment material was prepared by adding the desired amount (i.e. 60 wt % mirtazapine and 40 wt % EVA 33) of ingredients to a stainless steel drum after which the powder mixture was pre-mixed by rotating the drum on a Rhönrad at 47 rpm for 60 minutes. The powder mixture was subsequently fed to a Berstorff ZE25 co-rotating twin screw extruder and blend extruded at an extrusion temperature of 110° C. Blend extrusion resulted in strands in which mirtazapine was homogeneously dispersed in the EVA copolymer. The strands were subsequently granulated to inner compartment granulate. Prior to co-extrusion, the intermediate layer granulate was lubricated with 0.1 wt % magnesium stearate and homogenized in a stainless steel drum on a Rhönrad (barrel-hoop principle) with a fixed rotation speed of 47 rpm for 60 minutes.

The core granulate (EVA 28) was also lubricated with 0.1 wt % magnesium stearate and homogenized in stainless steel drum on a Rhönrad (barrel-hoop principle) with a fixed rotation speed of 47 rpm for 60 minutes.

The co-extrusion set-up consisted of a 15 mm skin extruder that processed the skin material, a 18 mm core extruder that processed the core material and an 18 mm inner compartment extruder that processed the i inner compartment granulate as delivered by the blend extruder. The melt flows were combined in a spinneret resulting in a three-layered skin-inner compartment-core fibre. The volume flow rate of all three melt flows was controlled by a set of separate spinning pumps. An extrusion temperature of approx. 105 to 115° C. and an extrusion rate of 1-2 m/min was used. Extrusion lead to a three-layered fibre with a diameter value of approx. 4 mm, a value of approx. 300 μm for the inner compartment and a skin thickness of approx. 30 μm. The fibre was cooled down to room temperature in a water bath and wound on a reel. The fibre was cut into 157 mm fibres using a semi-automatic cutter (Metzner) or by hand and subsequently the fibres were welded into a ring at 130° C.

TABLE 1
Dimensions of the mirtazapine rings produced comprising a core

| | | | | | | Cross- |
|---|---|---|---|---|---|---|
| | | Skin | Inner | | Concentration | sectional |
| | Skin | thickness | compartment | Inner compartment | drug | diameter |
| Batch | material | (μm) | layer material | layer thickness (μm) | (wt %) | (mm) |
| 1 | EVA 9 | 200 | EVA 33 | 576 | 40 | 4.1 |
| 2 | EVA 15 | 30 | EVA 33 | 576 | 40 | 4.0 |
| 6 | EVA 28 | 30 | EVA 33 | 576 | 40 | 4.0 |
| 7 | EVA 28 | 200 | EVA 33 | 659 | 40 | 4.1 |
| 10 | EVA 15 | 200 | EVA 33 | 341 | 60 | 4.1 |
| 11 | EVA 28 | 30 | EVA 33 | 341 | 60 | 4.1 |
| 12A | EVA 15 | 30 | EVA 18 | 1018 | 40 | 3.0 |
| 13 | EVA 33 | 30 | EVA 18 | 583 | 40 | 4.0 |
| 14 | EVA 15 | 30 | EVA 18 | 583 | 40 | 3.4 |
| 16 | EVA 15 | 30 | EVA 18 | 344 | 60 | 3.9 |
| 18 | EVA 33 | 30 | EVA 33 | 341 | 60 | 4.1 |
| 20 | EVA 28 | 30 | EVA 33 | 1068 | 60 | 4.0 |

Three-layered rings containing various materials and thicknesses for skin and inner compartment were manufactured (see Table 1). All batches had an EVA 28 core.

### In Vitro Release Rate of Core-Comprising Rings Containing Mirtazapine

In vitro release rate profiles of the vaginal rings were tested at 37° C. in water for 2 to 4 weeks. The results of the batches 7, 10 and 16 are given in FIG. 2 and of the batches 6, 11 and 18 in FIG. 3. The results in the figures clearly show that by varying the materials for skin and inner compartment, thickness of skin and inner compartment and concentration of drug (wt %) (Table 1) an average release on days 2-14 of approximately 7.5 mg/day (FIG. 2) and 15 mg/day (FIG. 3) can be achieved. Substantially constant release rates of approximately 25 mg/day are shown in FIG. 5, wherein the release results with batches 10 and 20 are compared.

### Steady State Release Rate of Core-Comprising Rings Containing Mirtazapine

TABLE 2
In vitro release rates of mirtazapine in water

Mirtazapine average release rate (mg/day)

| Batch | Day 2-14 | Day 2-28 | Day 14 | Day 28 |
|---|---|---|---|---|
| 1 | 1.4 | 1.4 | 1.3 | 1.2 |
| 2 | 12.4 | 9.4 | 8.7 | 5.9 |
| 6 | 16.3 | 11.3 | 9.4 | 5.8 |
| 7 | 8.0 | 6.7 | 6.6 | 5.0 |
| 10 | 7.8 | —* | 8.5 | —* |
| 11 | 15.1 | 13.9 | 16.2 | 6.5 |
| 12A | 8.0 | —* | 5.0 | —* |
| 13 | 8.3 | —* | 5.2 | —* |
| 14 | 6.8 | —* | 4.4 | —* |
| 16 | 7.1 | —* | 4.5 | —* |
| 18 | 15.3 | —* | 10.9 | —* |
| 20 | 23.7 | 24.7 | 25.7 | 23.5 |

—*= not determined

### Conclusions

The in vitro release rate profiles of the vaginal rings as given in FIGS. 2 and 3 show that, after a relatively high rate in the first 2-4 days, the release is prolonged at a constant release rate for periods up to and including 14 days. The initial high rate, that can be considered as a loading dose for fast attaining the desired plasma level in use, is clearly dependent on composition parameters and can be fine-tuned. An average release of day 2-14 of approximately 7.5 mg/day (Table 2: 7, 10 and 16) and 15 mg/day (Table 2: 6, 11 and 18) have been obtained.

A substantially constant release rate of approximately 25 mg/day is shown in FIG. 5.

### EXAMPLE 2

### Test for the Risk of Dose-Dumping

In an in vitro release study in water at 37° C. the mirtazapine release rate of a vaginal ring according to the invention is compared with a ring, cut into a rod with two open “ring-ends”. The in vitro results are depicted in FIG. 4. It is clearly shown that the release rate was not significantly affected, indicating that no dose-dumping occurred. Apparently the design of the device according to the invention inherently protects against dose-dumping problems of high-dose drug delivery systems comprising drugs like mirtazapine.

### EXAMPLE 3

### Preparation of Three-Layered Vaginal Rings Containing Risperidone

Preparation of three-layered vaginal rings consisted of several steps. First of all, an inner compartment granulate containing risperidone and EVA 33 copolymer was manufactured in a conventional way by pre-mixing, blend extrusion and lubrication with magnesium stearate. Secondly, a core material of EVA 28 was prepared by lubricating the as-supplied material. Subsequently, the inner compartment granulate, the core granulate and the skin material were co-extruded into a three-layered fibre. The fibre was cut to fibres of a specific length, as described below, after which the fibre ends were welded to a ring.

The inner compartment material was prepared by adding the desired amount of ingredients to a stainless steel drum after which the powder mixture was pre-mixed by rotating the drum on a Rhönrad at 47 rpm for 60 minutes. The powder mixture was subsequently fed to a Berstorff ZE25 co-rotating twin screw extruder and blend extruded at an extrusion temperature of 80° C. Blend extrusion resulted in strands in which risperidone was homogeneously dispersed in the EVA 33 copolymer. The strands were subsequently granulated to inner compartment granulate. Prior to co-extrusion, the intermediate layer granulate was lubricated with 0.1 wt % magnesium stearate and homogenized in a stainless steel drum on a Rhönrad (barrel-hoop principle) with a fixed rotation speed of 47 rpm for 60 minutes.

The core granulate EVA 28 was also lubricated with 0.1 wt % magnesium stearate and homogenized in stainless steel drum on a Rhönrad (barrel-hoop principle) with a fixed rotation speed of 47 rpm for 60 minutes.

The co-extrusion set-up consisted of a 15 mm skin extruder that processed the skin material, a 18 mm core extruder that processed the core material and an 18 mm inner compartment extruder that processed the inner compartment granulate as delivered by the blend extruder. The melt flows were combined in a spinneret resulting in a three-layered skin—inner compartment—core fibre. The volume flow rate of all three melt flows was controlled by a set of separate spinning pumps. An extrusion temperature of approx. 90° C. was used. Extrusion resulted in a three-layered fibre with a diameter value of approx. 4 mm. The fibre was cooled down to room temperature in a water bath and wound on a reel. The fibre was cut into 157 mm fibres and subsequently the fibres were welded into a ring at 110° C.

Three-layered rings containing various materials and thicknesses for skin and inner compartment were manufactured (see Table 3).

TABLE 3
Dimensions of the three-layered risperidone rings produced comprising
an EVA 28 core

| | | | Inner compartment | Concentration |
|---|---|---|---|---|
| | Skin | Skin thickness | layer thickness | drug |
| Batch | material | (μm) | (μm) | (wt %) |
| 1 | EVA 28 | 200 | 200 | 60 |
| 2 | EVA 28 | 200 | 400 | 60 |
| 3 | EVA 28 | 50 | 200 | 60 |
| 4 | EVA 28 | 50 | 400 | 60 |
| 5 | EVA 28 | 200 | 200 | 40 |
| 6 | EVA 28 | 200 | 400 | 40 |
| 7 | EVA 28 | 50 | 200 | 40 |
| 8 | EVA 28 | 50 | 400 | 40 |
| 9 | EVA 33 | 50 | 200 | 60 |
| 10 | EVA 15 | 50 | 200 | 60 |
| 11 | EVA 33 | 200 | 200 | 60 |
| 12 | EVA 15 | 200 | 200 | 60 |

### EXAMPLE 4

### In Vitro Release of Three-Layered Vaginal Rings Containing Risperidone

The in vitro release of three-layered vaginal rings containing risperidone was measured in water (buffered at pH 4.4) at 37° C. for at least 24 days. The dimensions of the risperidone rings produced comprising a core are reflected in Table 3.

The release rate of the three-layered vaginal rings can be tuned by choosing drug concentration, skin thickness and material. The average release rates for these batches are given in Table 4.

TABLE 4
Average in vitro release rates (AVG1 and AVG2) from three-layered
vaginal rings containing risperidone (n = 3)

| | AVG1: day 2-12 | AVG2: day 14-24 |
|---|---|---|
| Batch | (mg/day) | (mg/day) |
| 1 | 0.45 | 0.50 |
| 2 | 0.46 | 0.50 |
| 3 | 2.19 | 2.45 |
| 4 | 2.30 | 2.53 |
| 5 | 0.43 | 0.47 |
| 6 | 0.44 | 0.44 |
| 7 | 1.79 | 1.69 |
| 8 | 1.80 | 1.70 |
| 9 | 3.69 | 3.64 |
| 10 | 0.47 | 0.44 |
| 11 | 0.85 | 0.80 |

The influence of the vinyl acetate content of the skin material is shown in FIG. 6. The release rate is also influenced by the skin thickness of the vaginal ring as is shown in FIG. 7. FIG. 8 shows the influence of the drug concentration in the inner compartment layer. The figures show, that high release rates of risperidone with almost lacking initial burst release and substantially constant release rate can be obtained.

### EXAMPLE 5

### In Vitro Release of Three-Layered Rings Loaded with 20-70 Wt % of Mirtazapine (341 μm Intermediate Layer Thickness)

TABLE 5
Rings loaded with 20-70 wt % of mirtazapine were
made as specified in the following table:

| | | Intermediate layer | | | | | |
|---|---|---|---|---|---|---|---|
| | Core | | | | Drug load | Skin layer | |
| Batch | material | wt % | μm | material | (mg) | μm | material |
| A1 | EVA 28 | 20 | 341 | EVA 33 | 121 | 30 | EVA 28 |
| C1 | EVA 28 | 50 | 341 | EVA 33 | 325 | 30 | EVA 28 |
| D3 | EVA 28 | 60 | 341 | EVA 33 | 400 | 30 | EVA 28 |
| E1 | EVA 28 | 70 | 341 | EVA 33 | 479 | 30 | EVA 28 |

### Conclusion

The effect of drug load in the polymer is shown in FIG. 9. The release rate is more constant and substantial over an extended period of days with the rings loaded with 50 and 60 wt % of mirtazapine in the intermediate layer.

In Vitro Release of Three-Layered Rings Loaded with 40-70 wt % of Mirtazapine (682 μm Intermediate Layer Thickness)

TABLE 6
Rings loaded with 40-70 wt % of mirtazapine were made as
specified in the following table:

| | | Intermediate layer | | | | | |
|---|---|---|---|---|---|---|---|
| | Core | | | | Drug load | Skin layer | |
| Batch | material | wt % | μm | material | (mg) | μm | material |
| B4 | EVA 28 | 40 | 682 | EVA 33 | 459 | 30 | EVA 28 |
| D4 | EVA 28 | 60 | 682 | EVA 33 | 724 | 30 | EVA 28 |
| E2 | EVA 28 | 70 | 682 | EVA 33 | 868 | 30 | EVA 28 |

### Conclusion

The effect of drug load in the polymer is shown in FIG. 10 with a thicker intermediate layer of 682 μm. The release rate is more constant and substantial over an extended period of days with the rings loaded with 60 wt % of mirtazapine in the intermediate layer.

In Vitro Release of Three-Layered Rings Loaded with 60 wt % of Mirtazapine Having Skin Material EVA 28 (Batch D3) and EVA 15 (Batch D7).

TABLE 7
Rings were made as specified in the following table:

| | | Intermediate layer | | | | | |
|---|---|---|---|---|---|---|---|
| | Core | | | | Drug load | Skin layer | |
| Batch | material | wt % | μm | material | (mg) | μm | material |
| D3 | EVA 28 | 60 | 341 | EVA 33 | 400 | 30 | EVA 28 |
| D7 | EVA 28 | 60 | 341 | EVA 33 | 400 | 30 | EVA 15 |

### Conclusion

The effect of the use of EVA 15 in comparison to EVA 28 for the skin material is shown in FIG. 11. A more constant and still high release over an extended period is observed for the rings with EVA 15 skin material.

### EXAMPLE 6

### Comparison of Mirtazapine EVA Ring with Mirtazapine Silicone Ring

### Dimensions/Appearance

In FIGS. 12 and 13, the typical examples of a Silicone ring and an EVA ring are given. The outer diameter is identical, but the cross-sectional diameter of the Silicone ring is substantially higher (9 mm) as compared to an EVA ring (4 mm).

### Stiffness of the Rings

The stiffness of the ring is determined by means of a compression test.

A ring sample is positioned in its relaxed state (approx. 54 mm distance) between two holders. The two holders are moved with a speed of 50 mm/min to each other until the holders have a distance of approx. 21 mm. The forces to compress the ring are recorded at different compressions.

Table 8 gives the results of two representative batches of EVA rings and 4 different Silicone rings.

TABLE 8
Pressure test results of EVA ring (batch PD07.32137 (n = 4) and
PD07.32119 (n = 4)) and 4 different Silicone mirtazapine rings
(PD07.32228-PD07.32236).

| | Load at 10 mm | Load at 20 mm | Load at 30 mm |
|---|---|---|---|
| Batch | (N) | (N) | (N) |
| PD07.32137 | 1.3805 | 2.2728 | 4.5155 |
| PD07.32137 | 1.2017 | 1.9539 | 4.2025 |
| PD07.32137 | 1.6896 | 2.6437 | 4.7150 |
| PD07.32137 | 1.4804 | 2.3820 | 4.4535 |
| PD07.32119 | 1.1388 | 1.9476 | 3.9156 |
| PD07.32119 | 1.4795 | 2.2962 | 4.2307 |
| PD07.32119 | 1.6288 | 2.7136 | 5.0292 |
| PD07.32119 | 1.0540 | 1.7557 | 3.5217 |
| PD07.32128 | 3.2920 | 6.7030 | 16.928 |
| PD07.32132 | 4.1029 | 8.4870 | 20.622 |
| PD07.32134 | 2.7500 | 5.6155 | 13.352 |
| PD07.32136 | 2.8738 | 5.9380 | 14.828 |

### Conclusion

The Silicon ring is much stiffer than the EVA ring. The forces to compress the Silicon ring are approximately 3-4 times higher as compared to the EVA rings.

1-8. (canceled)

9. An extended release formulation comprising solid non-steroidal non-ionized hydrophilic drug having a molecular weight below 500, wherein the formulation is in the form of a vaginal device comprising:
(a) an inner compartment made of a thermoplastic polymer, wherein the thermoplastic polymer contains the drug, and wherein the drug is therapeutically effective in an amount below 60 mg daily; and
(b) a skin.

10. The formulation of claim 9, wherein the thermoplastic polymer contains 5 to 80 wt % of the drug.

11. The formulation of claim 9, wherein the skin is substantially continuous.

12. The formulation of claim 9, wherein the device is a ring.

13. The formulation of claim 9, wherein the thermoplastic polymer is ethylene vinyl acetate copolymer.

14. The formulation of claim 13, wherein the ethylene vinyl acetate copolymer has a vinyl acetate content in the range of 6 to 40%.

15. The formulation of claim 13, wherein the ethylene vinyl acetate copolymer has a vinyl acetate content of 28%.

16. The formulation of claim 15, wherein the drug has a solubility of at least 0.1 wt % in the ethylene vinyl acetate copolymer.

17. The formulation according to claim 9, wherein the inner compartment comprises a core, which does not contain the solid drug.

18. The formulation of claim 9, wherein the skin is made of ethylene vinyl acetate copolymer.
